(19) United States
(12) Patent Application Publication
(10) Pub. No.: US 2025/0128291 A1
(43) Pub. Date: Apr. 24, 2025

(54) Flexible Ultrasonic Sensor with Ultrasonic-Driven Liquid Metal as Conductive Material and Manufacturing Method Thereof

(71) Applicant: Harbin Institute of Technology, Harbin (CN)

(72) Inventors: Jia Zhang, Harbin (CN); Yuyang Li, Harbin (CN); Jiatai Chen, Harbin (CN); Xingqi Li, Harbin (CN)

(21) Appl. No.: 18/770,658

(22) Filed: Jul. 12, 2024

(30) Foreign Application Priority Data
Oct. 23, 2023 (CN) 202311373036.X

(51) Int. Cl. B06B 1/06; A61B 8/00; H10N 30/02; H10N 30/07; H10N 30/098; H10N 30/857; H10N 30/87; H10N 30/88

(52) U.S. Cl. CPC B06B 1/0688; A61B 8/4483; H10N 30/02; H10N 30/07; H10N 30/098; H10N 30/857; H10N 30/877; H10N 30/883; A61B 2562/12; B06B 2201/76

(57) ABSTRACT

A flexible ultrasonic sensor with an ultrasonic-driven liquid metal as a conductive material and a manufacturing method thereof are provided. The method includes: pumping a liquid metal into an inflow channel with a pre-embedded copper wire in an ultrasonic pumping mode; in a vacuum atmosphere, enabling a encapsulation film subjected to oxygen plasma treatment to slowly fall on the previously obtained material through a clamping apparatus; clamping a piezoelectric organic polymer by the obtained material and a liquid metal electrode bottom plate, enabling the piezoelectric organic polymer to be located at a central position through a positioning plate, and pasting a flexible encapsulation layer to a lower part of a whole device in the vacuum atmosphere; and sintering upper and lower layers of liquid metal electrodes by power ultrasound after heating to ensure electrical connectivity, thereby completing manufacturing of the flexible ultrasonic sensor.

### TECHNICAL FIELD

The present disclosure belongs to the technical field of flexible device manufacturing, and in particular to a manufacturing method of a flexible ultrasonic sensor with an ultrasonic-driven liquid metal as a conductive material.

### BACKGROUND

At present, almost all flexible ultrasonic sensors are based on the thinning of traditional rigid ultrasonic probes and the addition of flexible materials. As shown in FIG. 1A, components inevitably use some traditional rigid materials, such as copper electrodes for electrical interconnection and lead zirconate titanate (PZT) series piezoelectric ceramics. However, defects of a flexible device composed of the aforementioned components are obvious. The substantial difference in the elastic modulus of each component can lead to the loss of electrical connectivity due to repeated deformation during routine and daily applications.

Liquid metal (LM), based on indium-gallium alloy, presents a novel electrode form that can effectively address the flexibility issues of electrodes in wearable devices. And the device is assisted with a flexible piezoelectric polymer with excellent performance (such as PVDF and related copolymers thereof), thereby realizing the fully-flexible and highly-deformable flexible ultrasonic sensing method. The manufactured device is easy to attach to multiple complex curved surfaces, and thus the application scenarios of the flexible sensing technology in daily health monitoring could be widely expanded. With the rapid development of liquid metals, an ultrasonic-driven method is introduced to inject LM into electrode channel, and then assisted by ultrasonic sintering, devices, electrodes, etc. can be manufactured on substrates with large flexibility and high ductility, thereby meeting requirements for flexibility, ductility and stability in routine and daily applications.

### SUMMARY

In view of the problems existing in the prior art, the present disclosure employs an ultrasonic driving mode for injecting indium gallium alloy, and provides a manufacturing method of a flexible ultrasonic sensor with an ultrasonic-driven liquid metal as a conductive material.

The present disclosure is implemented by the following technical solution:

A flexible ultrasonic sensor with a liquid metal as the conductive material is provided.

A sensor includes: an organic piezoelectric polymer, a flexible electrode layer containing an inflow channel and an outflow channel on one side, an electrode layer with a liquid metal completely on one side, a liquid metal, and an outermost flexible encapsulation layer, where
the organic piezoelectric polymer is PVDF and related polymers thereof, and a selected thickness of the organic piezoelectric polymer is determined based on an ultrasonic penetration depth required for different organs as detecting targets;
the flexible electrode layer containing an inflow channel and an outflow channel on one side serves as a backing layer which is close to the organic piezoelectric polymer, is configured to provide electrical connectivity, to transmit input current or to measure an output signal of a sensor, and is attached to the organic piezoelectric polymer to enable the organic piezoelectric polymer to receive excitation and perceive external stimulation as well as to generate corresponding electroacoustic and acoustoelectric responses, thereby providing a certain assistance in suppressing backward transmission of ultrasound and helping to improve a bandwidth of a device;
the electrode layer with a liquid metal completely on one side is configured to transmit current or perform other specific functions, and the liquid metal is located on an other side of the organic piezoelectric polymer to ensure effective current conduction while providing a common-ground wiring form;
the liquid metal conducts current between the electrode layer and the organic piezoelectric polymer to trigger and measure the electroacoustic and acoustoelectric responses of the organic piezoelectric polymer; and
the outermost flexible encapsulation layer is configured to protect and encapsulate the entire flexible ultrasonic sensor to protect internal components and materials.

A manufacturing method of a flexible ultrasonic sensor with an ultrasonic-driven liquid metal as a conductive material is provided.

The method specifically includes the following steps:
step 1: pumping a liquid metal into an inflow channel with a pre-embedded copper wire with the help of ultrasonic pumping;
step 2: in a vacuum atmosphere, allowing an encapsulation film previously subjected to oxygen plasma treatment to be slowly placed on a material obtained from step 1 through a clamping apparatus;
step 3: clamping a piezoelectric organic polymer by the material obtained from step 2 and a liquid metal bottom electrode, allowing the piezoelectric organic polymer to be located at a central position through a positioning plate, and pasting a flexible encapsulation layer to a lower part of a whole device in the vacuum atmosphere; and

step 4: sintering upper and lower layers of liquid metal electrodes by 600 W-720 W power ultrasound after slowly heating to a room temperature so as to ensure electrical connectivity, thereby completing manufacturing of the flexible ultrasonic sensor.

An electronic equipment is provided, including a memory and a processor. The memory stores a computer program. When the processor performs the computer program, the steps of the aforementioned method are implemented.

A computer-readable storage medium is provided for storing a computer instruction. When the computer instruction is executed by a processor, the steps of the aforementioned method are implemented.

### Beneficial Effects of the Present Disclosure

The pore inflow resistance designed by the present disclosure is small, and the residual liquid metal in the outflow channel can be used as a backing layer to improve bandwidth performance in the ultrasonic sensor. Finally, the flexible encapsulation film subjected to plasma treatment is employed to assist in clamping and sealing an opening in the vacuum atmosphere, so as to eliminate residual gases as much as possible.

Furthermore, the flexible piezoelectric material is selected as PVDF or related polymers P(VDF-TrFE) thereof. All of them have excellent piezoelectric performance, etc. Compared with traditional piezoelectric materials (such as hard piezoelectric ceramics), they have a wider frequency response range and better mechanical characteristics. Moreover, they are easy to form acoustic impedance matching with human tissues and have the optimal characteristics of lighter and easy bending design.

Compared with technologies such as photolithography, vacuum deposition and chemical deposition, the flexible device manufactured as mentioned in the present disclosure has advantages of high speed, low cost, etc., and can be manufactured in large quantities. As shown in FIG. 1B, the manufactured device also has highly stable flexibility, and transparent and lightweight manufacturing of products can be achieved.

By using the method and related materials described in the present disclosure, an electrode with a line width as small as 5 μm can be manufactured, enabling the manufacturing of large-scale high arrays. Under high-power ultrasound-assisted sintering, the finally obtained conductivity of the liquid metal can be greater than 10⁸ S·m⁻¹. The double-sided electrode manufacturing method provided by the present disclosure improves the overall flexibility of the device to ensure that the performance of the device is not affected by extreme working conditions such as repeated bending, while serving as a necessary acoustic matching and backing layer. Furthermore, the method described in the present disclosure eliminates manual operations as much as possible, thereby ensuring the efficiency and improving the yield, while also improving the manufacturing consistency across different processing batches.

### BRIEF DESCRIPTION OF FIGURES

FIG. 1A-B shows comparative diagrams of longitudinal sectional views of flexible ultrasonic sensors with the traditional flexible ultrasound and liquid metal as conductive materials, where FIG. 1A shows a longitudinal sectional view of sub-units of a flexible traditional rigid ultrasonic sensor, and FIG. 1B shows a longitudinal sectional view of units of a flexible ultrasonic sensor with a liquid metal as a conductive material;

FIG. 2A shows an exploded view of a flexible ultrasonic sensor with a liquid metal as a conductive material, and FIG. 2B shows a top view of an outflow direction of the metal liquid of an upper layer of an electrode; and

FIG. 3 shows a flow diagram of a manufacturing method of a flexible ultrasonic sensor with an ultrasonic-driven liquid metal as a conductive material.

### DETAILED DESCRIPTION

The technical solutions in examples of the present disclosure will be clearly and completely described below with reference to accompanying drawings in the examples of the present disclosure. Obviously, the described examples are only a part rather than all of the examples of the present disclosure. All other examples obtained by those of ordinary skill in the art based on the examples in the present disclosure without creative efforts shall fall within the scope of protection of the present disclosure.

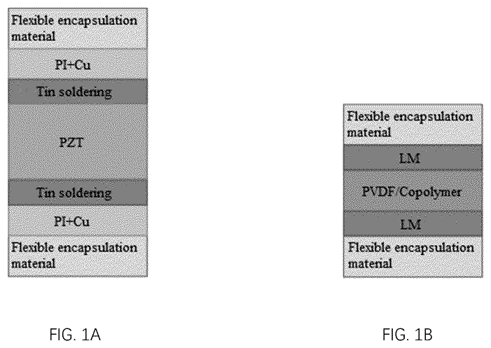

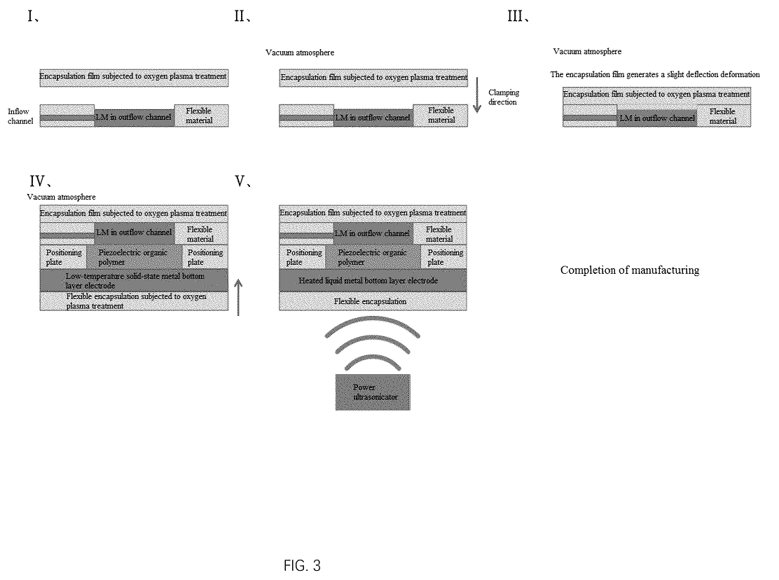

With reference to FIG. 1A-B and FIG. 3,
a flexible ultrasonic sensor with an ultrasonic-driven liquid metal as a conductive material is provided.

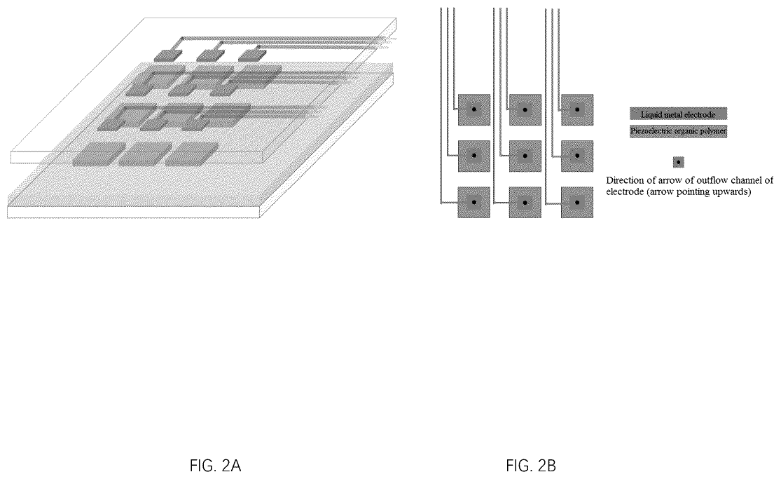

A sensor includes: an organic piezoelectric polymer, a flexible electrode layer containing an inflow channel and an outflow channel on one side (backing layer), an electrode layer with a liquid metal completely on one side (matching layer), a liquid metal (LM), and an outermost flexible encapsulation layer, where exploded views of materials of each part are shown in FIG. 2A.

The organic piezoelectric polymer is PVDF and related polymers thereof, and a thickness of the organic piezoelectric polymer is determined based on different organs as detecting targets. Since a frequency constant is a determined value of a certain piezoelectric material, the piezoelectric polymer described in the present disclosure has multiple center frequencies, i.e., multiple possible thicknesses, such as high center frequencies for superficial organs or low center frequencies for deep organs.

FIG. 2B illustrates fine cylindrical inflow channels embedded in flexible materials and outflow channels with openings above square tensile bodies of an upper layer of the flexible electrode.

The inflow channel and the outflow channel are configured to guide a fluid to allow the fluid to enter or flow out of a device, the inflow channel is configured to pump the liquid metal under ultrasonic drive to form an electrode, and the outflow channel is configured to discharge the excessive liquid metal to ensure a completeness of infusion.

The inflow channel is cylindrical, is positioned on one side of the device, and is configured to pump the liquid metal (indium gallium alloy) under ultrasonic drive to form an electrode. The outflow channel shown in FIG. 2B is square structure, and the liquid alloy in the square outflow channel also serves as a backing layer of the ultrasonic device. A copper wire is pre-embedded on a periphery to facilitate a subsequent connection of a BCN interface.

The inflow channel is positioned below a spatial position of the device, the outflow channel is positioned above the spatial position of the device, the liquid metal pumped under ultrasonic drive is easy to fill a whole pathway due to an action of gravity, and the excessive liquid metal is discharged to ensure the completeness of infusion.

The flexible electrode layer containing an inflow channel and an outflow channel on one side serves as a backing layer which is close to the organic piezoelectric polymer, is configured to provide electrical connectivity, to transmit input current or to measure an output signal of a sensor, and is attached to the organic piezoelectric polymer to enable the organic piezoelectric polymer to receive excitation and perceive external stimulation as well as to generate corresponding electroacoustic and acoustoelectric responses, thereby providing some assistance in suppressing backward transmission of ultrasound and helping to improve a bandwidth of the device.

The electrode layer with a liquid metal completely on one side employs a common-ground design, and the electrode also serves as a matching layer in the acoustic device. The electrode layer is configured to transmit current or perform other specific functions. The liquid metal is located on an other side of the organic piezoelectric polymer to ensure effective current conduction while providing a common-ground wiring form. Furthermore, as part of the acoustic device, an appropriate thickness can be designed to facilitate transmission of ultrasound to the human side.

The liquid metal is configured to match the flexible electrode layer and conduct current, is positioned between the electrode layer and the flexible encapsulation layer, and conducts current between the electrode layer and the organic piezoelectric polymer to trigger or measure the response of the organic piezoelectric polymer.

The outermost flexible encapsulation layer is configured to protect and encapsulate the entire flexible ultrasonic sensor to protect internal components and materials. The outermost flexible encapsulation layer surrounds the flexible electrode layer and the liquid metal electrode layer to encapsulate the flexible electrode layer and the liquid metal electrode layer inside, and provides flexible performance, so that the entire device is suitable for attachment to different curved surfaces or for use in different application scenarios. This design helps to ensure the performance and stability of the device.

In the multi-element sensor array form, the inflow port requires finer inflow channels, and each array has at most one bend, resulting in minimal inflow resistance. Furthermore, due to the larger space in the outflow channels, the main volume of liquid metal is stored above the piezoelectric organic polymer. During the actual manufacturing process, slight overflow of the liquid metal can be observed, ensuring the completeness of the infusion.

The configuration of the piezoelectric ceramic array of the present disclosure is not limited to three-by-three arrangement depicted in the figures. In high-precision imaging, more piezoelectric ceramic subunits can be employed as needed.

The electrode layer with a liquid metal completely on one side employs a common-ground electrode, which also serves as the matching layer, and the thickness can be determined according to the following formula:

t=N·λ/4

where t represents the thickness, N represents a magnification factor which usually ranges from 1.16 to 1.18 (in order to achieve better acoustic matching in practical experiments), and λ represents a wavelength of ultrasound in a medium at a working frequency and represents a wavelength of ultrasound propagating through an indium-gallium alloy, as presented in this disclosure.

FIG. 2A illustrates fine cylindrical inflow channels embedded in flexible materials and outflow channels with openings above square tensile bodies of an upper layer of flexible electrode. The indium gallium alloy is pumped under ultrasonic drive to form an electrode. Moreover, the alloy in the square outflow channel may also serve as a backing layer of the ultrasonic device. A copper wire is pre-embedded on the periphery to facilitate the subsequent connection of a BCN interface.

A manufacturing method of a flexible ultrasonic sensor with an ultrasonic-driven liquid metal as a conductive material is provided. The focus of present disclosure is the manufacturing and assembling processes of upper and lower layers of flexible liquid metal electrodes of the device, as shown in FIG. 3.

The method specifically includes the following steps:

Step 1: A liquid metal is pumped into an inflow channel with a pre-embedded copper wire with the help of ultrasonic pumping.

In step 1, the liquid metal should be observed to overflow slightly to ensure that the channels are fully filled, thereby guaranteeing the completeness of infusion, as shown in the first sub-figure in FIG. 3.

Step 2: In a vacuum atmosphere, an encapsulation film previously subjected to oxygen plasma treatment is slowly placed on a material obtained from step 1 through a clamping apparatus.

In step 2, the encapsulation film will naturally generate a certain degree of deflection deformation when in contact with the material obtained from step 1, and the plasma-treated encapsulation film ensures that the flexible material part has stronger adhesion, thereby further ensuring that the liquid is completely sealed and gases are excluded as much as possible, as shown in the second and third sub-figures in FIG. 3.

Step 3: A piezoelectric organic polymer is clamped by the material obtained from step 2 and the liquid metal bottom electrode, the piezoelectric organic polymer is located at a central position through a positioning plate, and a flexible encapsulation layer is pasted to a lower part of a whole device in the vacuum atmosphere.

In step 3, the liquid metal bottom electrode is first subjected to low-temperature curing treatment to ensure curing and provide stable support. The flexible encapsulation layer, before adhesion, undergoes plasma treatment of the surrounding flexible encapsulation parts using masking to cover the metal part, enhancing its adhesion and cohesion.

Step 4: The upper and lower layers of liquid metal electrodes are sintered by 600 W-720 W power ultrasound after slowly heating to a room temperature so as to ensure electrical connectivity, thereby completing manufacturing of the flexible ultrasonic sensor.

In present disclosure, the main components of the liquid metal include 75 wt. % of gallium and 25 wt. % of gallium.

A PDMS (Sylgard 184, Dow Corning) prepolymer is prepared from a polymeric base and a curing agent in a ratio of 10:1, and is used as a flexible encapsulation material in the description of this patent.

During the application of power ultrasound to the device, the temperature of the sample is maintained at a constant 15° C. using a cold water bath.

An electronic equipment is provided, including a memory and a processor. The memory stores a computer program. When the processor executes the computer program, the steps of the aforementioned method are implemented.

A computer-readable storage medium is provided for storing a computer instruction. When the computer instruction is executed by a processor, the steps of the aforementioned method are implemented.

The memory in the examples of the present disclosure may be a volatile memory or a non-volatile memory, or may include both the volatile memory and the non-volatile memory, where the non-volatile memory may be a read only memory (ROM), a programmable ROM (PROM), an erasable PROM (EPROM), an electrically EPROM (EEPROM), or a flash memory; and the volatile memory may be a random access memory (RAM), which serves as an external cache. By way of exemplary but not restrictive illustration, many forms of RAM are available, such as a static RAM (SRAM), a dynamic RAM (DRAM), a synchronous DRAM (SDRAM), a double data rate SDRAM (DDR SDRAM), an enhanced SDRAM (ESDRAM), a synchlink DRAM (SLDRAM), and a direct rambus RAM (DR RAM). It should be noted that the memory in the method described in the present disclosure is intended to include but not limited to these memories and any other suitable types of memories.

The manufacturing method of the flexible ultrasonic sensor with an ultrasonic-driven liquid metal as a conductive material, provided by the present disclosure, is introduced in detail above. The principles and implementations of the present disclosure are illustrated. The descriptions of the above examples are only intended to help understand the method of the present disclosure and the core idea thereof. At the same time, for those of ordinary skill in the art, according to the idea of the present disclosure, there will be changes in specific implementations and application scope. In light of the aforementioned considerations, it is important to note that the content of this specification should not be construed as a limitation on the present disclosure.

What is claimed is:

1. A flexible ultrasonic sensor with a liquid metal as a conductive material, wherein
the flexible ultrasonic sensor comprises: an organic piezoelectric polymer, a flexible electrode layer containing an inflow channel and an outflow channel on one side, an electrode layer with a liquid metal completely on one side, a liquid metal, and an outermost flexible encapsulation layer, wherein
the organic piezoelectric polymer is PVDF and related polymers thereof, and a selected thickness of the organic piezoelectric polymer is determined based on an ultrasonic penetration depth required for different organs as detecting targets;
the flexible electrode layer containing an inflow channel and an outflow channel on one side serves as a backing layer which is close to the organic piezoelectric polymer, is configured to provide electrical connectivity, to transmit input current or to measure an output signal of the flexible ultrasonic sensor, and is attached to the organic piezoelectric polymer to enable the organic piezoelectric polymer to receive excitation and perceive external stimulation as well as to generate corresponding electroacoustic and acoustoelectric responses;
the electrode layer with a liquid metal completely on one side is configured to transmit current or perform other specific functions, and the liquid metal is located on another side of the organic piezoelectric polymer to ensure effective current conduction while providing a common-ground wiring form;
the liquid metal conducts current between the electrode layer and the organic piezoelectric polymer to trigger and measure electroacoustic and acoustoelectric responses of the organic piezoelectric polymer; and
the outermost flexible encapsulation layer is configured to protect and encapsulate entirety of the flexible ultrasonic sensor to protect internal components and materials.

2. The flexible ultrasonic sensor according to claim 1, wherein
the inflow channel and the outflow channel are configured to guide a fluid to allow the fluid to enter or flow out of the flexible ultrasonic sensor, the inflow channel is configured to pump the liquid metal under ultrasonic drive to form an electrode, and the outflow channel is configured to discharge excessive liquid metal to ensure a completeness of infusion; and
the inflow channel is positioned below a spatial position of the device, the outflow channel is positioned above the spatial position of the device, and the liquid metal pumped under ultrasonic drive is easy to fill a whole pathway due to an action of gravity.

3. The flexible ultrasonic sensor according to claim 2, wherein
the inflow channel is cylindrical and is located on one side of the device, and a copper wire is pre-embedded on a periphery for a subsequent connection of a BCN interface; and
the outflow channel is a square structure, and a liquid alloy in the outflow channel also serves as the backing layer of the flexible ultrasonic sensor.

4. The flexible ultrasonic sensor according to claim 3, wherein
the electrode layer with a liquid metal completely on one side employs a common-ground design, the electrode also serves as a matching layer in the flexible ultrasonic sensor, and a thickness is determined according to the following formula:
$$t = N \cdot \frac{λ}{4}$$
wherein t represents the thickness, N represents a magnification factor which usually ranges from 1.16 to 1.18, and λ represents a wavelength of ultrasound in a medium at a working frequency.

5. A manufacturing method of a flexible ultrasonic sensor with an ultrasonic-driven liquid metal as a conductive material, wherein
the method specifically comprises the following steps:
step 1: pumping a liquid metal into an inflow channel with a pre-embedded copper wire with help of ultrasonic pumping;
step 2: in a vacuum atmosphere, allowing an encapsulation film previously subjected to oxygen plasma treatment to be slowly placed on a material obtained from step 1 through a clamping apparatus;
step 3: clamping a piezoelectric organic polymer by the material obtained from step 2 and a liquid metal bottom electrode, allowing the piezoelectric organic polymer to be located at a central position through a positioning plate, and pasting a flexible encapsulation layer to a lower part of a whole device in the vacuum atmosphere; and
step 4: sintering upper and lower layers of liquid metal electrodes by 600 W-720 W power ultrasound after slowly heating to room temperature so as to ensure electrical connectivity, thereby completing manufacturing of the flexible ultrasonic sensor.

6. The manufacturing method according to claim 5, wherein
in step 1, the liquid metal is be observed to overflow slightly to ensure a completeness of infusion.

7. The manufacturing method according to claim 6, wherein
in step 2, the encapsulation film naturally generates a certain degree of deflection deformation when in contact with the material obtained from step 1, and the encapsulation film is subjected to plasma treatment to ensure a tightness of bonding and a sealing of an encapsulation space, so as to eliminate adverse effects of residual gases in a circuit on the circuit in the vacuum atmosphere.

8. The manufacturing method according to claim 7, wherein
in step 3, the liquid metal bottom electrode is first subjected to a low-temperature curing treatment; and the flexible encapsulation layer is first subjected to a plasma treatment before pasting.

9. An electronic equipment, comprising a memory and a processor, the memory storing a computer program, wherein when the processor executes the computer program, the steps of the method according to claim 5 is implemented.

10. A computer-readable storage medium for storing a computer instruction, wherein when the computer instruction is executed by a processor, the steps of the manufacturing method according to claim 5 is implemented.
